(19) United States
(12) Patent Application Publication
(10) Pub. No.: US 2022/0226276 A1
(43) Pub. Date: Jul. 21, 2022

(54) COMPOSITIONS FOR DELIVERY OF LADOSTIGIL

(71) Applicant: Yissum Research Development Company of The Hebrew University of Jerusalem Ltd., Jerusalem (IL)

(72) Inventor: Marta WEINSTOCK-ROSIN, Jerusalem (IL)

(73) Assignee: Yissum Research Development Company of The Hebrew University of Jerusalem Ltd., Jerusalem (IL)

(21) Appl. No.: 17/614,530

(22) PCT Filed: May 28, 2020
(86) PCT No.: PCT/IL2020/050598

§ 371 Date: Nov. 28, 2021

Related U.S. Application Data
(60) Provisional application No. 62/853,194, filed on May 28, 2019.

(51) Int. Cl. A61K 31/27; A61P 25/28; A61K 9/00

(52) U.S. Cl. CPC A61K 31/27; A61K 9/0053; A61K 9/0031; A61P 25/28

(57) ABSTRACT

The invention provides compositions comprising ladostigil, being a colonic delivery composition, including methods and uses thereof.

### BACKGROUND OF THE INVENTION

Oxidative stress resulting from impaired mitochondrial function and resulting in the excess production of reactive oxygen species plays a key role in the etiology of Alzheimer's disease (AD) (Sayre et al. 2008; Smith et al. 2005). Impaired mitochondrial function in AD (Valla et al. 2001) is accompanied by microglial activation that increases the release of pro-inflammatory cytokines (Maccioni et al. 2009; Mangialasche et al. 2009). Oxidative stress and microglial activation are already seen in the brain of subjects with mild cognitive impairment (MCI) prior to the development of AD (Butterfield et al. 2007; Okello et al. 2009; Yasuno et al. 2012; Pardo et al. 2017).

Ladostigil (Scheme 1) 6-(N-ethyl, N-methyl, carbamyloxy)-N propargyl-1(R)-aminoindan, tartarate can protect neuronal cells against damage induced by oxidative stress (Weinstock et al. 2001) and stimulate the activity of antioxidant enzymes (BarAm et al. 2009). It can also reduce the release of pro-inflammatory cytokines from activated microglia (Panarsky et al. 2012). Chronic administration of ladostigil to aging rats prevents the decline in recognition and spatial memory (Weinstock et al. 2011, 2013) and the alterations in microglial morphology and their associated genes in a brain region specific manner (Shoham et al. 2018).

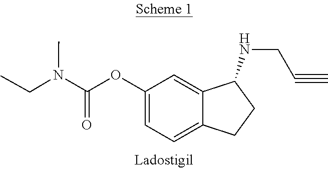 [SMILES: C#CCN[C@@H]1CCc2ccc(OC(=O)N(C)CC)cc21]

In a phase 2 study in human subjects with MCI there were indications that ladostigil may slow the decline in episodic memory, whole brain and hippocampal volume and the progression to dementia (Schneider et al. 2019).

Ladostigil is well absorbed from the gastro intestinal tract and can be detected in the blood within 15 minutes after oral administration in humans and rodent species. However, the inter subject variability in these blood levels at peak times after administration is very high with coefficients of variation of more than 100% irrespective of the dose administered. (Table 1). This is undesirable and could exacerbate any variability in the response to treatment by ladostigil of potential patients. The wide inter individual variation in blood levels may explain why the therapeutic effect of the drug in the clinical trial was not more pronounced.

TABLE 1
Concentration of ladostigil in plasma of mice rats and human subjects after a single oral dose

| Time | Mice | | Rats | | Rats | | Humans | | Humans | |
|---|---|---|---|---|---|---|---|---|---|---|
| (min) | (10 mg/kg) | | (1 mg/kg) | | (5 mg/kg) | | (50 mg) | | (100 mg) | |
| 15 | 118 ± 160 | (136) | 18.7 ± 15.0 | (80) | 53.7 ± 52.3 | (99) | 3.74 ± 6.49 | (174) | 9.30 ± 9.09 | (98) |
| 30 | 58.6 ± 39.7 | (68) | 12.3 ± 9.2 | (75) | 36.4 ± 37.1 | (102) | 18.03 ± 28.3 | (156) | 34.8 ± 58.7 | (169) |
| 60 | 11.9 ± 9.7 | (82) | 6.7 ± 3.9 | (88) | 8.3 ± 5.1 | (81) | 8.29 ± 6.20 | (75) | 97.6 ± 115.5 | (118) |
| 120 | 8.7 ± 10.6 | (122) | 1.60 ± 0.92 | (58) | 4.9 ± 4.4 | (109) | 2.88 ± 2.71 | (94) | 44.4 ± 33.8 | (76) |

| | | | | | | | |
|---|---|---|---|---|---|---|---|
| 180 | NT | BL | 2.4 ± 2.1 | (93) | BL | 15.3 ± 12.3 | (80) |

Data are expressed as ng/ml, mean±STD. Lado=ladostigil. NT=not tested; BL=below level of detection. Experiments were performed in male ICR mice, weighing 25-30 g (Moradov et al. 2015) and Wistar rats weighing 250-270 gm. There were at least 10 animals at each time point. The lowest concentration of ladostigil detectable was 50 pg/ml. The measurements in humans were carried out by Parexel in 6 normal healthy subjects aged 18-40. Measurements of ladostigil were made by Biogal Pharmaceutical Co. Ltd. (Debrecen, Hungary), using a validated LC/MS/MS method. The lowest concentration of ladostigil detectable was 2 ng/ml. ( ) represents the coefficient of variation.

There is therefore a need for a novel method of delivery of ladostigil that will reduce the wide inter individual variation in blood levels after oral dosing.

### SUMMARY OF THE INVENTION

The present invention provides a composition comprising ladostigil, wherein said composition is a colonic delivery composition. In another aspect the invention provides a composition comprising ladostigil formulated for colonic delivery. The invention further provides a colonic-targeted composition comprising ladostigil.

When referring to ladostigil it should be understood to encompass the compound represented in Scheme 1 above, and including any pro-drugs thereof, metabolites thereof, enantiomers, enantiomer mixtures, conjugated acid/base forms thereof and any combinations thereof. Ladostigil is a neuroprotective agent being effective in the treatment of neurodegenerative disorders like Alzheimer's disease, Lewy body disease, and Parkinson's disease. It acts as a pseudo reversible acetylcholinesterase and butyrylcholinesterase inhibitor, and an irreversible monoamine oxidase B inhibitor. Ladostigil also has antidepressant effects and may be useful for treating depression and anxiety often seen in such diseases as well.

In addition to its neuroprotective properties, seen at lower concentrations than those inhibiting these enzymes, ladostigil enhances the expression of neurotrophic factors like GDNF and BDNF and may be capable of reversing some of the damage seen in neurodegenerative diseases via the induction of neurogenesis.

When referring to compositions formulated for colonic delivery and/or colonic delivery composition and/or colonic-targeted composition it should be understood to encompass any formulation that allows for drug delivery specifically through the colon, without (or with minimal) absorption in the upper gastrointestinal (GI) tract. This type of administration allows for a higher concentration of the drug to reach the colon with minimal systemic absorption. The colonic contents have a longer retention time (up to 5 days), and the colonic mucosa is capable of facilitating the absorption of drugs, making this organ an ideal site for drug delivery. A drug can be delivered to the colon via the oral, or the rectal route.

Thus, in some embodiments, a composition of the invention is an oral composition (i.e. said composition is administered through the oral cavity and/or mucosal membrane thereof). In other embodiments, a composition of the invention is a rectal composition (i.e. said composition is administered through the rectal cavity and/or mucosal membrane thereof).

In some embodiments, a colonic-delivery composition of the invention is in the form selected from a colon-specific prodrug carrier, a colon-specific biodegradable delivery system, a matrix-based system, a time release system, a bio adhesive system, a multi-particulate system, a poly-saccharide based delivery system, having a colon targeted coating, osmotic/pressure control delivery system, pulsincap system and any combinations thereof.

When referring to colon-specific prodrug carrier it should be understood to encompass any inactive derivatives of the drug molecule which release the active ingredient once they are hydrolyzed by enzymes such as those in the colon. In order to optimize drug delivery specific to the colon, the extent of this hydrolysis should be minimal in the upper portions of the gastrointestinal tract and much more extensive in the colon.

When referring to colon-specific biodegradable delivery system it should be understood to relate to any type of delivery system encompassing the drug molecule being designed to degrade specifically in the presence of colonic bacteria and/or enzyme produced by colonic bacteria. The colon contains many species of anaerobic bacteria which obtain their energy by fermenting substrates such as polymers which have not yet been digested. Bacteroides, eubacteria, clostridia, enterococci, and enterobacteria are some examples of these colon-specific species, and they produce numerous enzymes such as glucuronidase, xylosidase, nitroreductase, and azoreductase to ferment polymers.

When referring to matrix-based system it should be understood to relate to any delivery system that embeds the drug molecule in at least one polymer matrix trapping it therein and releasing it in the colon. These matrices can be pH-sensitive or biodegradable.

A time release system includes formulations that are based on the drug molecule being released in the colon after a specified amount of time. This approach is dependent on the transit time through the small intestine, which is typically between 3 and 4 h.

A bio adhesive system allows a formulation to remain in contact within the colon, for a long period of time to assist in targeted absorption of the drug molecule therein. In some embodiments, polymers which have been used in bioadhesive formulations include but are not limited to polycarbophils, polyurethanes, polyethylene oxide and any combinations thereof.

A multi-particulate system refers to a delivery system formulated into particulate matter having smaller particle size that can reach the colon quickly since they pass through the GI tract more easily. Microspheres are one example of a multi-particulate system that can be loaded with a drug for colonic delivery. Microspheres that are prepared using biodegradable components can be taken up by macrophages.

When referring to poly-saccharide based delivery system it should be understood to relate to any type of polysaccharides (including, but not limited to pectin, chitosan, chondroitin sulfate, galactomannan, amylose and any combinations thereof) that are degraded by the colonic enzymes and are harmless to the organisms. The polysaccharides are used in thin film coatings of the drug molecule but also include matrix systems and compression coatings. Pectin, for example, is a hydrophilic polysaccharide which can modify drug release due to its gelling ability. An insoluble polymer such as ethyl cellulose (EC) is often mixed with the pectin in the coating layer to help reduce water permeability and protect the drug core.

When referring to a composition having a colon targeted coating it should be understood to relate to a composition incorporating the drug molecule in at least one pH-sensitive polymer allow for delayed release by protecting the active ingredient from the acidic pH of the stomach and proximal small intestine. These polymers then break down in the more basic pH of the terminal ileum, thus providing a targeted drug delivery to the colon. Some none limiting examples of commonly used pH-sensitive polymers in the design of colon-targeted drug delivery systems include methacrylic-acid based polymers, also known as Eudragit®. Enteric-soluble polymers are resistant to dissolution in the acidic environment of the stomach but can dissolve at the higher pH values of the intestine. Additionally, coatings with these polymers are designed to be relatively thick to prolong their dissolution and provide a controlled or an extended drug release. In addition to enteric-soluble polymers, acid-soluble polymers can also be used in colon-targeted formulations.

Compression-coating (tablet-in-a-tablet), also known as B dry coating is a tablet coating technique where the core tablet (containing the drug) is coated with a coating excipient (powder) on a tablet press.

Rupturable film coatings allow a drug to be released after undergoing a timed disruption caused by hydrostatic pressure within the core. Since these polymeric films are permeable, an influx of water and subsequent swelling of the hydrophilic polymers can initiate the disruption. Permeable film coatings allow water to pass through and dissolve the drug containing core, but the polymeric coating itself is insoluble.

These coatings do not rupture after exposure to an aqueous medium because they are permeable and resistant to dissolution. Additionally, the materials within these coatings do not expand after an influx of water. Since it takes time for the drug to diffuse out from the core after dissolving, this results in a lag phase before drug release occurs.

Another type of time-dependent coating is a semipermeable film coating which is similar to permeable coatings in that they are permeable to water. However, these coatings are impermeable to solutes. Water moves into the tablet core of the formulation due to osmotic pressure, and when the hydrostatic pressure within the system exceeds the osmotic pressure after a programmed lag phase, small orifices in the outer membrane allow the drug which has dissolved in the aqueous medium to be pumped out.

When referring to osmotic/pressure control delivery system, relate to delivery systems that take advantage of peristaltic motions that cause the luminal pressure of the large intestine to increase more than that of the small intestine because its contents are more viscous due to the reabsorption of water. These systems allow for drugs to be delivered to the colon rather than the small intestine due to higher colonic pressure.

The OROS-CT is an example of a system regulated by osmotic pressure. It consists of a hard gelatin capsule which dissolves in the pH of the small intestine and allows water to enter the unit. This then causes it to swell and the drug is forced out (3). Within each capsule there can be as many as 5-6 units, and each unit is surrounded by a drug impermeable enteric coating which prevents water from entering in the acidic environment of the stomach. However, this coating dissolves and the water enters once the capsule enters the higher pH of the small intestine. Within the enteric coating there is a semipermeable membrane which encompasses an osmotic push compartment as well as a drug compartment. The water causes the push compartment to swell and forms a gel in the drug compartment that is forced out of an orifice through the membrane next to the drug compartment. The rate at which the drug flows out depends on the rate at which water enters. To prevent drug release in the small intestine, these systems can also be designed such that there is a lag time between when the enteric coating dissolves and the drug is released.

The integration of a timed-release system with pH-sensitive properties can be beneficial in achieving colon-targeted delivery. A pulsincap system is one example of a formulation that utilizes both these techniques. The system consists of a water insoluble capsule body containing the drug, a hydrogel plug which seals the opened end of this capsule body and a water soluble cap which covers the hydrogel plug. Additionally, the capsule is coated with an acid insoluble film coating which prevents the drug from being released in the stomach. The hydrogel plug begins swelling when this enteric coating dissolves in the small intestine. The swelling of the plug allows for a lag time before the drug is released and the amount of lag time depends on the length of the plug and the extent at which it is inserted.

In some embodiments, said active molecule, ladostigil is further formulated for sustained release, immediate release, modified release, delayed-release dosage, extended release, prolonged release, long-acting release and any combinations thereof.

In some embodiments, said composition of the invention is a liquid dosage form (e.g., enemas). In some other embodiments, said composition of the invention is a solid dosage form (e.g., suppositories, capsules, and tablets). In further embodiments, said composition of the invention is a semi-solid dosage form (e.g., gels, foams, ointments and creams).

In some embodiments, said composition of the invention is an enema. In these embodiments, said dosage form comprises the drug (ladostigil) in solution, suspension, or emulsion. Furthermore, in these embodiments, said enema is administered using a disposable contained (such as a plastic squeeze bottles with an extended tip for rectal insertion).

In some embodiments, said composition of the invention is a suppository. In these embodiments, said composition is a solid dosage form comprises the drug (ladostigil) that is either dispersed or dissolved in a suitable base. Drugs are typically mixed with the suppository excipients during manufacturing to form a homogenous system. Suppositories are generally composed of either a lipophilic base (e.g., cocoa butter, coconut oil, hydrogenated vegetable oils, and hard fats) or hydrophilic base (e.g., glycerinated gelatin and polyethylene glycols). Lipophilic bases are immiscible with body fluids and readily melt at body temperature to release the drug on the mucosal surface, whereas hydrophilic bases need to dissolve in the physiological fluids for drug release.

In some embodiments, said suppository is a hollow-type suppository. Hollow-type suppository contains a hollow space in the center that is filled with the drug in solid, liquid, or semi-solid form. The solid outer shell of the suppository can be composed of hydrophilic or lipophilic base materials and can incorporate other constituents to confer additional release properties, such as muco-adhesion and sustained release.

In other embodiments, said suppository is a dimple-type suppository. Dimple-type suppository has one or more dimples on the surface where drugs are embedded. It was proposed that concentrating the drug to a limited area on the surface of the suppository would lead to a higher rate of drug release and absorption when administered into the rectum. In addition, limiting the drug concentration toward the surface of the suppository increases its contact with the rectal mucosal surface and creates a concentration gradient for passive absorption of the drug across the mucosa.

In some embodiments, said composition of the invention is in the form of a gel. In other embodiments said composition of the invention is in the form of a foam. Gel and foam formulations generally require the use of an applicator that has to be filled with the drug formulation prior to dose administration. Rectal gels are semi-solid formulations that contain a solvent trapped within a polymer network to create a viscous consistency. Viscosity of the gel can be modified by the addition of co-solvents (e.g., glycerin and propylene glycol) and electrolytes.

In some embodiments, a composition of the invention is a liquid suppository. In some embodiments, liquid suppositories comprise thermosensitive polymers, mucoadhesive polymers or a combination of thermosensitive and mucoadhesive polymers. Poloxamers are the commonly used thermosensitive polymers in pharmaceutical formulation. Mucoadhesive polymers (e.g., carbopol, sodium alginate, polycarbophil, hydroxypropyl methylcellulose, hydroxyethyl cellulose, and methylcellulose) have been used in combination with thermosensitive polymers to improve gel strength and muco-adhesion. It should be noted that cellulose ether polymers (e.g., hydroxypropyl methylcellulose, hydroxyethyl cellulose, and methylcellulose) also possess controlled release characteristics. These hydrogels are able to swell over time, which would also allow the encapsulated drug to be released at a continuous rate.

In some embodiments, a composition of the invention is a foam. Foams are generally considered a colloidal dosage form, with a hydrophilic liquid continuous phase containing a foaming agent and a gaseous dispersion phase distributed throughout. Following rectal administration, they transition from a foam state to a liquid or semi-solid state on the mucosal surface. The structure of the foam is affected by parameters such as concentration and nature of the foaming agent, pH and temperature of the system, and viscosity of the liquid phase. Foaming agents are amphiphilic substances that are important for foam generation and stabilization. The molecules contain hydrophilic components that are soluble in the aqueous phase and hydrophobic components that form micelles to minimize contact with the aqueous phase.

In some embodiments, said composition is a nanoparticulate system for rectal delivery.

In another aspect the invention provides a composition of the invention for use in the treatment of Alzheimer's disease, including conditions and symptoms thereof.

In a further aspect, the invention provides a method of treating Alzheimer's disease, including conditions and symptoms thereof; said method comprising administering a composition comprising ladostigil, wherein said composition is a colonic delivery composition.

In a further aspect, the invention provides a composition comprising ladostigil, wherein said composition is a colonic delivery composition, for use in the treatment of mild cognitive impairment, including conditions and symptoms thereof.

In a further aspect, the invention provides a method of treating mild cognitive impairment, including conditions and symptoms thereof; said method comprising administering a composition comprising ladostigil, wherein said composition is a colonic delivery composition.

When relating to mild cognitive impairment (MCI) it should be understood to relate to a condition of a subject that is between the expected cognitive decline of normal aging and the more serious decline of dementia. It can involve at least one of problems with memory, language, thinking and judgment that are greater than normal age-related changes.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Example 1: A Comparison of Oral and Rectal Administration of Ladostigil in Rats

Methods: Male Wister rats weighing 250-270 g were used for these experiments. Ladostigil was administered by gavage at a dose of 5 mg/kg (volume 0.25-0.27 ml). In other rats, the same dose of ladostigil was administered via a cannula gently inserted into the rectum and pushed forward to reach the colon. The rats were anesthetized with isofluorane and sacrificed by decapitation at 0, 15, 30, 60 or 120 min after ladostigil administration. The brain was removed, and the cortex carefully dissected and stored at −80° C. until further analysis. Blood was collected in heparinized Eppendorf tubes, centrifuged at 4° C., 20,800 g for 10 min and the plasma was stored at −80° C. until further analysis.

Samples were prepared for LC-MS analysis after precipitating the proteins in plasma and cortical extracts with methanol as described in Moradov et al. (2015). Briefly, rivastigmine 750 ng/ml (internal standard) was added to 50 μl of plasma or supernatant after brain homogenization and ladostigil was extracted with HPLC grade MeOH. 75 μl of double distilled water (DDW) was added to 150 μl of the supernatant to give a final ratio of DDW:MeOH 1:1. The samples were filtered through 0.45 μM GHP membranes and injected into the LC-MS machine. LC-MS/MS analysis was also performed as described in Moradov et al. (2015).

Ladostigil and rivastigmine were detected by an AB Sciex (Framingham, Mass., USA) Triple Quad™ 5500 mass spectrometer in positive ion mode by electrospray ionization (ESI) and a multiple reaction monitoring (MRM) mode of acquisition. Data acquisition and analysis were performed on a Dell Optiplex 960 computer with Analyst 1.6.2 software distributed by AB Sciex. Quantitative calibration (0-100 ng/ml) was performed before every batch of samples. The calibration curve (y=a+bx) was obtained by linear least-squares regression of the measured peak area (y) versus the concentration added to the biological matrix (x). The limit of quantification (LOQ) of ladostigil in plasma and cortex was 50 pg/ml.

TABLE 2
Concentration of ladostigil in plasma and brain (cortex)
after oral or rectal administration of 5 mg/kg in rats

| Time | Oral administration | | | | Rectal administration | | | |
|---|---|---|---|---|---|---|---|---|
| (m) | Plasma | | Cortex | | Plasma | | Cortex | |
| 15 | 53.7 ± 52.3 | (99) | 17.9 ± 18.5 | (103) | 60.6 ± 23.4 | (39) | 14.3 ± 6.7 | (47) |
| 30 | 36.4 ± 37.1 | (102) | 9.3 ± 7.5 | (81) | 47.2 ± 25.6 | (54) | 11.9 ± 5.5 | (46) |
| 60 | 8.3 ± 5.1 | (81) | 6.7 ± 5.2 | (86) | 33.7 ± 19.0 | (56)* | 9.3 ± 4.8 | (52) |
| 120 | 4.9 ± 4.4 | (109) | 4.9 ± 5.4 | (110) | 14.1 ± 8.7 | (62)* | 7.4 ± 5.1 | (69) |
| 180 | 2.4 ± 2.1 | (93) | 2.9 ± 2.8 | (97) | 13.9 ± 5.4 | (39)* | 2.8 ± 1.4 | (50) |

Data are expressed as mean±STD, ng/ml for plasma, ng/g for cortex in groups of at least 15 rats for plasma and 10, for cortex. Significantly higher than concentration after oral treatment *p<0.05 (2 tailed). ( )=coefficient of variation.

Development for administration in human subjects: For colonic administration of ladostigil in human subjects, a formulation that transfers the drug intact to the colon is used, from which it would reach the systemic circulation. Colonic delivery has additional advantages: (a) the typical, specific enzyme activity in this organ can be exploited for a continuous erosion of saccharide polymers at a predesigned rate to yield zero order release kinetics; (b) the uniform construct of the colon epithelium, together with the long residence time in its lumen, creates a homogenous reservoir-like organ with a constant drug input (zero order release) into the circulation. It has already been shown that PK profile after colonic drug administration resembles that obtained after intravenous infusion (but at an order of magnitude lower).

Colonic formulations: The design of the orally administered colonic formulations takes into consideration two major prerequisites: (a) stability in the lumen of the small intestine (i.e. the ability to pass, intact, until arriving at the ileo-cecal junction), (b) slow erosion in the lumen of the colon to result in slow release of the entrapped ladostigil at a predetermined rate. The formulation is adapted from previously developed, IP protected techniques that have also been validated for safety. If necessary, a mixture of a protective coating and a colon-specific biodegradable matrix might be used, examples of which are shown (Amidon et al 2015; Handali et al; 2018).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### REFERENCES

1. Amidon S, Brown J E, Dave V S. Colon-targeted oral drug delivery systems: design trends and approaches. AAPS Pharm Sci Tech 16:731-741, 2015.
2. Bar-Am O, Weinreb O, Amit T, Youdim M B. The novel cholinesterase-monoamine oxidase inhibitor and antioxidant, ladostigil, confers neuroprotection in neuroblastoma cells and aged rats. J Mol Neurosci 37:135-145, 2009.
3. Butterfield D A, Reed T T, Perluigi M, De Marco C, Coccia R, Keller J N, Markesbery W R, Sultana R. Elevated levels of 3-nitrotyrosine in brain from subjects with amnestic mild cognitive impairment: implications for the role of nitration in the progression of Alzheimer's disease. Brain Res 1148:243-248, 2007.
4. Goelman G, Ilinca R, Zohar I, Weinstock M. Functional connectivity in prenatally stressed rats with and without maternal treatment with ladostigil, a brain-selective monoamine oxidase inhibitor, Eur J Neurosci 40: 2734-2743, 2014.
5. Handali S, Moghimipour E, Rezaei M, Kouchak M, Ramezani Z, Dorkoosh F A. In vitro and in vivo evaluation of coated capsules for colonic delivery. J Drug Deliv Sci Tech 47:492-498, 2018.
6. Maccioni R B, Rojo L E, Fernandez J A, Kuljis R O. The role of neuroimmunomodulation in Alzheimer's disease. Ann N Y Acad Sci 1153:240-246, 2009.
7. Mangialasche F, Polidori M C, Monastero R, Ercolani S, Camarda C, Cecchetti R, Mecocci P. Biomarkers of oxidative and nitrosative damage in Alzheimer's disease and mild cognitive impairment. Ageing Res Rev 8:285-305, 2009.
8. Moradov D, Finkin-Groner E, Bejar C, Sunita P, Schorer-Apelbaum D, Barasch D, Nemirovski A, Cohen M, Weinstock M. Dose-limiting inhibition of acetylcholinesterase by ladostigil results from the rapid formation and fast hydrolysis of the drug-enzyme complex formed by its major metabolite, R-MCPAI. Biochem Pharmacol 94:164-172, 2015.
9. Okello A, Edison P, Archer H A, Turkheimer F E, Kennedy J, Bullock R, Walker Z, Kennedy A, Fox N, Rossor M, Brooks D J. Microglial activation and amyloid deposition in mild cognitive impairment: a PET study. Neurology 72(1):56-62, 2009.
10. Panarsky R, Luques L, Weinstock M. Anti-inflammatory effects of ladostigil and its metabolites in aged rat brain and in microglial cells. J Neuroimmune Pharmacology 7: 488-498, 2012.
11. Pardo J, Abba M C, Lacunza E, Francelle L, Morel G R, Outeiro T F, Goya R G. Identification of a conserved gene signature associated with an exacerbated inflammatory environment in the hippocampus of aging rats. Hippocampus 27:435-449, 2017.
12. Phillip A. K. et al. Colon Targeted Drug Delivery Systems: A Review on Primary and Novel Approaches. Oman Medical Journal 25:70-78, 2010.
13. Sayre L M, Perry G, Smith M A. Oxidative stress and neurotoxicity. Chem Res Toxicol 21:172-188, 2008.
14. Schneider L, et al. Low-dose ladostigil for mild cognitive impairment: a phase 2 placebo-controlled clinical trial. Neurology in press
15. Shoham S, Linial M, Weinstock M. Age-induced spatial memory deficits in rats are correlated with specific brain region alterations in microglial morphology and gene expression. J Neuroimmune Pharmacology doi.org/10.1007/s 11481-018-9817-2, 2018.
16. Smith M A, Nunomura A, Lee H G, Zhu X, Moreira P I, Avila J, Perry G. Chronological primacy of oxidative stress in Alzheimer disease. Neurobiol Aging 26:579-580, 2005.
17. Valla J, Berndt J D, Gonzalez-Lima F. Energy hypometabolism in posterior cingulate cortex of Alzheimer's patients: superficial laminar cytochrome oxidase associated with disease duration. J Neurosci 21:4923-4930, 2001.
18. Weinstock M, Bejar C, Schorer-Apelbaum D, Panarsky R, Luques L, Shoham S. Dose-dependent effects of ladostigil on microglial activation and cognition in aged rats. J Neuroimmune Pharmacology 8:345-355, 2013.
19. Weinstock M, Kirschbaum-Slager N, Lazarovici P, Bejar C, Youdim M B H, Shoham S. Neuroprotective effects of novel cholinesterase inhibitors derived from rasagiline as potential anti-Alzheimer drugs. Ann New York Acad Sci, 939:148-161, 2001.
20. Weinstock M, Luques L, Poltyrev T, Bejar C, Shoham S. Ladostigil prevents age-related glial activation and spatial memory deficits in rats. Neurobiol Aging 32:1069-1078, 2011.
21. Yasuno F, Kosaka J, Ota M, Higuchi M, Ito H, Fujimura Y, Nozaki S, Takahashi S, Mizukami K, Asada T, Suhara T. Increased binding of peripheral benzodiazepine receptor in mild cognitive impairment-dementia converters measured by positron emission tomography with [(¹¹)C]DAA1106. Psychiatry Res 203:67-74, 2012.

What is claimed is:

1. A composition comprising ladostigil, being a colonic delivery composition.

2. A composition comprising ladostigil formulated for colonic delivery.

3. A composition according to claim 1 or 2, being an oral composition.

4. A composition according to claim 1 or 2, being a rectal composition.

5. A composition according to any one of the preceding claims, being selected from a colon-specific prodrug carrier, a colon-specific biodegradable delivery system, a matrix-based system, a time release system, a bio adhesive system, a multi-particulate system, a poly-saccharide based delivery system, having a colon targeted coating, osmotic/pressure control delivery system, pulsincap system and any combinations thereof.

6. A composition according to any one of claims 1 to 5, for use in the treatment of mild cognitive impairment, including conditions and symptoms thereof.

7. A composition according to any one of claims 1 to 5, for use in the treatment of Alzheimer's disease, including conditions and symptoms thereof.
